(19) United States
(12) Patent Application Publication
(10) Pub. No.: US 2020/0159045 A1
(43) Pub. Date: May 21, 2020

(54) SYSTEM AND METHOD FOR ULTRASONIC BLINK DETECTION

(71) Applicant: Johnson & Johnson Vision Care, Inc., Jacksonville, FL (US)

(72) Inventors: Donald Scott Langford, Melbourne, FL (US); Adam Toner, Jacksonville, FL (US)

(21) Appl. No.: 16/197,281

(22) Filed: Nov. 20, 2018

(51) Int. Cl. G02C 7/08; A61B 8/08; G02C 7/04

(52) U.S. Cl. CPC G02C 7/083; G02C 7/04; A61B 8/08

(57) ABSTRACT

A ophthalmic lens having an electronic system is described herein for detecting blinks using at least one transducer emitting a sound pressure wave(s) to be reflected from the inside of the eyelid when the wearer is blinking or otherwise has his/her eyelid closed. The ophthalmic lens in further embodiments may communicate with a second ophthalmic lens to confirm the detection of a blink. In at least one embodiment, the ophthalmic lens includes at least one ultrasound module having at least one transducer such as a pair of transmit and receive transducers, a transceiver transducer or a plurality of transducers. The ultrasound module includes additional components for the creation and reception of the sound pressure wave(s). The ophthalmic lenses may be contact lenses or intraocular lenses.

### BACKGROUND OF THE INVENTION

### 1. Field of the Invention

The present invention relates to a powered or electronic ophthalmic lens, and more particularly, to a powered or electronic ophthalmic lens having an ultrasound module for use in detecting a blink.

### 2. Discussion of the Related Art

As electronic devices continue to be miniaturized, it is becoming increasingly more likely to create wearable or embeddable microelectronic devices for a variety of uses. Such uses may include monitoring aspects of body chemistry, administering controlled dosages of medications or therapeutic agents via various mechanisms, including automatically, in response to measurements, or in response to external control signals, and augmenting the performance of organs or tissues. Examples of such devices include glucose infusion pumps, pacemakers, defibrillators, ventricular assist devices and neurostimulators. A new, particularly useful field of application is in ophthalmic wearable lenses and contact lenses. For example, a wearable lens may incorporate a lens assembly having an electronically adjustable focus to augment or enhance performance of the eye. In another example, either with or without adjustable focus, a wearable contact lens may incorporate electronic sensors to detect concentrations of particular chemicals in the precorneal (tear) film. The use of embedded electronics in a lens assembly introduces a potential requirement for communication with the electronics, for a method of powering and/or re-energizing the electronics, for interconnecting the electronics, for internal and external sensing and/or monitoring, and for control of the electronics and the overall function of the lens.

The human eye has the ability to discern millions of colors, adjust easily to shifting light conditions, and transmit signals or information to the brain at a rate exceeding that of a high-speed internet connection. Lenses, such as contact lenses and intraocular lenses, currently are utilized to correct vision defects such as myopia (nearsightedness), hyperopia (farsightedness), presbyopia and astigmatism. However, properly designed lenses incorporating additional components may be utilized to enhance vision as well as to correct vision defects.

Conventional contact lenses are polymeric structures with specific shapes to correct various vision problems as briefly set forth above. To achieve enhanced functionality, various circuits and components have to be integrated into these polymeric structures. For example, control circuits, microprocessors, communication devices, power supplies, sensors, actuators, light-emitting diodes, and miniature antennas may be integrated into contact lenses via custom-built optoelectronic components to not only correct vision, but to enhance vision as well as provide additional functionality as is explained herein. Electronic and/or powered ophthalmic lenses may be designed to provide enhanced vision via zoom-in and zoom-out capabilities, or just simply modifying the refractive capabilities of the lenses. Electronic and/or powered contact lenses may be designed to enhance color and resolution.

The proper combination of devices could yield potentially unlimited functionality; however, there are a number of difficulties associated with the incorporation of extra components on a piece of optical-grade polymer. In general, it is difficult to manufacture such components directly on the lens for a number of reasons, as well as mounting and interconnecting planar devices on a non-planar surface. It is also difficult to manufacture to scale. The components to be placed on or in the lens need to be miniaturized and integrated onto just 1.5 square centimeters of a transparent polymer while protecting the components from the liquid environment on the eye. It is also difficult to make a contact lens comfortable and safe for the wearer with the added thickness of additional components.

In addition, because of the complexity of the functionality associated with a powered lens and the high level of interaction between all of the components comprising a powered lens, there is a need to coordinate and control the overall operation of the electronics and optics comprising a powered ophthalmic lens. Accordingly, there is a need for a system to control the operation of all of the other components through wearer input via blinks and/or monitoring the wearer that is safe, low-cost, and reliable, has a low rate of power consumption and is scalable for incorporation into an ophthalmic lens. Accordingly, there exists a need for a means and method for receiving input from the wearer of at least one ophthalmic lens and/or monitoring the wearer while the ophthalmic lens is being worn/used.

### SUMMARY OF THE INVENTION

In at least one embodiment, an ophthalmic lens system includes: an ophthalmic lens; at least one ultrasound module in the ophthalmic lens, the at least one ultrasound module having at least one transmit transducer and at least one receive transducer; at least one system controller in electrical communication with the at least one ultrasound module, the at least one system controller configured to provide an initiation signal to the at least one ultrasound module to cause the at least one ultrasound module to produce at least one sound pressure wave, and the at least one system controller configured to receive any signal from the at least one ultrasound module representative of a reflection sound pressure wave; and a timing circuit in electrical communication with the system controller, the timing circuit configured to produce a timing signal when the system controller is activated, and wherein the at least one of the system controller and the at least one ultrasound module configured to determine if eyelid closure indicative of blink has occurred based on receipt of the reflection sound pressure wave within a predetermined time (or sampling) period and/or having an amplitude above a predetermined strength threshold, and the at least one system controller configured to act upon a determination that at least one blink has occurred.

In a further embodiment, the ophthalmic lens system further includes an actuator in electrical communication with the system controller, the actuator configured to receive an output signal from the system controller, and the actuator configured to at least one of store data at least temporarily and produce an action based on the output signal. In a further embodiment to the above embodiments, the at least one ultrasound module is configured to deactivate the at least one receive transducer after a predetermined sampling period. In a further embodiment to the above embodiments, a plurality of ultrasound modules is distributed around the periphery of the ophthalmic lens, the distribution of ultrasound modules providing for at least one ultrasound module to be covered by an eyelid closure. In a further embodiment to the above embodiments, the ophthalmic lens system according to claim 1, further including a power supply in electrical communication with the at least one system controller.

In a further embodiment to the above embodiments, the at least one system controller includes a memory storing at least one blink template for use by the at least one system controller based on an operation state of the system controller to determine if a wearer has provided an instruction. Further to the previous embodiment, the at least one system controller is configured to select at least one blink template stored in the memory. Further to the previous embodiment, the memory stores at least one blink mask to allow for the wearer to deviate from a desired intentional blink sequence for blink detection. Further to the previous three embodiments, the memory is configured to store at least one blink determination.

In a further embodiment to the above embodiments, the at least one system controller determines lid closure to occur when the at least one system controller receives an indication of the reflected sound pressure wave from the at least one ultrasound module within at most≤50 μs from transmission of the sound pressure wave by the at least one transmit transducer.

In a further embodiment to the above embodiments, the ophthalmic system further including a second ophthalmic lens; and wherein the at least one system controller configured to provide at least one output signal to be sent to the second ophthalmic lens in response to a determination that a blink has occurred. In a further embodiment, the at least one ultrasound module further configured to provide communications to the second ophthalmic lens.

In at least one embodiment, an ultrasonic method for blink detection by an ophthalmic lens wherein the ophthalmic lens includes an ultrasound module in electrical communication with a system controller, the method includes: creating a pulse to drive a transducer in the ultrasound module; emitting a sound pressure wave from the transducer; producing a signal by the ultrasound module in response to any received sound pressure wave within 50 μS of the sound pressure wave being emitted by the transducer; determining by at least one of the ultrasound module and the system controller whether the signal produced is within a predetermined blink threshold; and storing the blink determination in a memory. In a further embodiment, the method further including comparing stored blinks to at least one of a blink template and a blink mask by the system controller. In a further embodiment to the above method embodiments, the memory includes a register for storing a plurality of blink determinations. In a further embodiment to the above method embodiments, the ophthalmic lens contains at least two ultrasound modules at different positions around the lens periphery; and the method further including: activating the ultrasound module with a most varied output signal in response to the sound pressure wave, and deactivating by the at least one system controller the non-selected ultrasound modules.

In at least one embodiment, an ultrasonic method for blink detection by an ophthalmic lens, the method includes: creating a sound pressure wave by at least one transducer; receiving a sound pressure wave by at least one transducer; producing an output in response to the received sound pressure wave; comparing a series of outputs to a predefined blink template and/or a mask by at least one of an ultrasound module and a system controller; and if at least one set of sampled data matches the blink template and/or the mask, instructing an actuator to perform an action by the system controller. In a further embodiment, the method further including changing an operation state of the ophthalmic lens by the system controller when at least one set of outputs matches the predefined blink template and/or mask. In a further embodiment to the embodiments of this paragraph, the method further including comparing the number of blinks, the duration of the blinks in a given time (or sampling) period, and a time between blinks in a given time (or sampling) period to a stored set of samples representative of one or more predetermined intentional blink sequences to receive instructions from the wearer. In a further embodiment to the embodiments of this paragraph, there are two ophthalmic lenses, the method further including: communicating at least the detection of a blink from one ophthalmic lens to the other ophthalmic lens; determining whether both ophthalmic lenses detected a blink at substantially the same time.

In a further embodiment, the method further includes determining if at least one set of lid closures correspond to at least one predetermined intentional blink sequences including allowance for deviations in the durations of the blinks from the durations of the blinks in the one or more predetermined intentional blink sequences by using at least one mask. In a further embodiment to the above embodiments when there is communication between the lenses, determinations are made regarding a blink when both lenses are agreement that a blink occurred. In a further embodiment, the blink determinations are used as part of other monitoring of the wearer.

Further to the previous embodiments, the ophthalmic lens includes an intraocular lens and/or a contact lens.

Further to any of the embodiments above, a message sent by the system controller of the first ophthalmic lens uses a predefined protocol. Further to any of the embodiments above, the message sent by the system controller of the first ophthalmic lens includes instructions for the second ophthalmic lens to perform a predefined function. Further to any of the embodiments above, the message sent by the system controller of the first ophthalmic lens includes sensor readings from at least one sensor on the first ophthalmic lens.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 illustrates a contact lens having at least one ultrasound module in accordance with at least one embodiment of the present invention.

FIG. 2 illustrates a contact lens having at least one ultrasound module and a system controller having a register in accordance with at least one embodiment of the present invention.

FIG. 3 illustrates a contact lens having at least one ultrasound module and a timing circuit in accordance with at least one embodiment of the present invention.

FIG. 4 illustrates an ultrasound module in accordance with at least one embodiment of the present invention.

FIG. 5 illustrates an ultrasound module with one transducer and a multiplexer in accordance with at least one embodiment of the present invention.

FIG. 6 illustrates an ultrasound module with a charge pump and an envelope detector in accordance with at least one embodiment of the present invention.

FIG. 7 illustrates an ultrasound module with one transducer and a multiplexer in accordance with at least one embodiment of the present invention.

FIG. 8 illustrates an ultrasound module with one transducer and a multiplexer in accordance with at least one embodiment of the present invention.

FIG. 9 illustrates a diagrammatic representation of an electronic insert, including a pair of transducers, for a powered contact lens in accordance with at least one embodiment of the present invention.

FIG. 10 illustrates a diagrammatic representation of an electronic insert, including a transducer, for a powered contact lens in accordance with at least one embodiment of the present invention.

FIG. 11 illustrates a diagrammatic representation of a plurality of ultrasound modules/transducers spaced around a powered contact lens in accordance with at least one embodiment of the present invention.

FIG. 12 illustrates an ultrasound module with a plurality of transmit/receive transducer pairs or transceiver transducers in accordance with at least one embodiment of the present invention.

FIGS. 13 and 14 illustrate methods of determining blinks using ultrasound time of flight measurements in accordance with embodiments of the present invention.

FIG. 15 illustrates a method of determining blinks using ultrasound strength measurements in according with at least one embodiment of the present invention.

FIG. 16 illustrates a pair of contact lenses in accordance with at least one embodiment of the present invention.

### DETAILED DESCRIPTION OF THE EMBODIMENTS

Conventional contact lenses are polymeric structures with specific shapes to correct various vision problems as briefly set forth above. To achieve enhanced functionality, various circuits and components may be integrated into these polymeric structures. For example, control circuits, microprocessors, communication devices, power supplies, sensors, ultrasound modules, and miniature antennas may be integrated into contact lenses via custom-built optoelectronic components to not only correct vision, but to enhance vision as well as provide additional functionality as is explained herein. Electronic and/or powered contact lenses may be designed to provide enhanced vision via zoom-in and zoom-out capabilities, or just simply modifying the refractive capabilities of the lenses. Electronic and/or powered contact lenses may be designed to enhance color and resolution. In addition, ultrasound modules built into the lenses may be utilized to detect blink patterns, for example to change operational state, allow the wearer to provide instructions to the contact lens, and/or monitor blink activity of the wearer.

The powered or electronic ophthalmic lens in at least one embodiment includes the necessary elements to monitor the wearer with or without elements to correct and/or enhance the vision of the wearer with one or more of the above described vision defects or otherwise perform a useful ophthalmic function. The electronic ophthalmic lens may have a variable-focus optic lens, an assembled front optic embedded into an ophthalmic lens or just simply embedding electronics without a lens for any suitable functionality. The electronic lens of the present invention may be incorporated into any number of ophthalmic lenses as described above. Ophthalmic lenses include contact lenses and intraocular lenses. Intraocular lenses may also incorporate the various components and functionality described herein. However, for ease of explanation, the disclosure will focus on an electronic contact lens intended for single-use daily disposability.

The present invention may be employed in a powered ophthalmic lens having an electronic system, which actuates a variable-focus optic or any other device or devices configured to implement any number of numerous functions that may be performed. The electronic system includes one or more batteries or other power sources, power management circuitry, one or more sensors, clock generation circuitry, control algorithms and circuitry, and lens driver circuitry. The complexity of these components may vary depending on the required or desired functionality of the lens.

In at least one embodiment, control of the powered ophthalmic lens may be accomplished via feedback or control signals directly from the wearer such as through the wearer blinking. For example, ultrasound modules built into the lens may detect blinks, blink patterns, eyelid closures, and/or eye movement depending upon the configuration of the ultrasound modules, which in at least one embodiment include a transmit ultrasound transducer and at least one receive ultrasound transducer, a combination transmit/receive ultrasound transducer, or a combination passive transmit/receive backscatter ultrasound transducer. Based upon the pattern or sequence of blinks and/or movement, the powered ophthalmic lens may change operation state such as change focus of the contact lens. A further alternative is that the wearer has no control over operation of the powered ophthalmic lens and data is collected about the wearer based on eyelid position.

Because of the complexity of the functionality associated with a powered lens and the high level of interaction between all of the components comprising a powered lens, there is a need to coordinate and control the overall operation of the electronics and/or optics comprising a powered ophthalmic lens. Accordingly, there is a need for a system to control the operation of all of the other components and provide communication between the lenses that is low-cost and reliable, has a low rate of power consumption, and is scalable for incorporation into an ophthalmic lens.

In at least one embodiment, a sound pressure wave produced at the transmit ultrasound transducer propagates (or emits) from the contact lens into the field of view. Objects in the field of view will reflect and/or scatter the sound pressure wave. There is a finite amount of time that passes between the generation of the transmitted sound pressure wave and the return of the reflected signal. This time is determined by the speed of sound in air (typically 343 meters/second) and two times the distance to the object. Two times the distance to the object is used to account for the initial time it takes the sound pressure wave to travel from the transmit ultrasound transducer to the object and the time it takes the reflected wave to travel back to the receive ultrasound transducer.

A blink detection algorithm is a component of the system controller which detects characteristics of blinks, for example, is the lid open or closed, the duration of the blink, the inter-blink duration, the number of blinks in a given time period, and the length of lid closure. The algorithm in accordance with the present invention relies on sampling for reflected sound pressure waves from the eyelid at a certain sample rate. Pre-determined blink patterns are stored and compared to the recent samples. When patterns match, the blink detection algorithm may trigger activity in the system controller, for example, to activate the lens driver to change the refractive power of the lens or to change the operation state of the lens. The blink detection algorithm further distinguishes between the pre-determined blink patterns and the eyelid movements associated with drowsiness or sleep onset has occurred.

Blinking is the rapid closing and opening of the eyelids and is an essential function of the eye. Blinking protects the eye from foreign objects, for example, individuals blink when objects unexpectedly appear in proximity to the eye. Blinking provides lubrication over the anterior surface of the eye by spreading tears. Blinking also serves to remove contaminants and/or irritants from the eye. Normally, blinking is done automatically, but external stimuli may contribute as in the case with irritants. However, blinking may also be purposeful, for example, for individuals who are unable to communicate verbally or with gestures can blink once for yes and twice for no.

The blink detection algorithm and system of the present invention utilizes blinking patterns that cannot be confused with normal blinking response. In other words, if blinking is to be utilized as a means for controlling an action, then the particular pattern selected for a given action cannot occur at random; otherwise inadvertent actions may occur. As blink speed and/or frequency may be affected by a number of factors, including fatigue, concentration, boredom, eye injury, medication and disease, blinking patterns for control purposes preferably account for these and any other variables that affect blinking. The average length of involuntary blinks is in the range of about one hundred (100) to four hundred (400) milliseconds with the eyelid closing motion typically being much faster than the eyelid opening motion. Average adult men and women blink at a rate of ten (10) involuntary blinks per minute, and the average time between involuntary blinks is about 0.3 to seventy (70) seconds. Eyelid movements may also indicate other conditions such as drowsiness as the eyelids have a general trend towards closing over a period of time or are closed for a period of time indicating that the wearer is asleep.

An embodiment of the blink detection algorithm may be summarized in the following steps:

1. Define an intentional “blink sequence” that a user will execute for positive blink detection or that is representative of sleep onset.

2. Sample for reflected sound pressure waves at a rate consistent with detecting the blink sequence and rejecting involuntary blinks.

3. Compare the history of sampled reflected sound pressure waves to the expected “blink sequence,” as defined by a blink template of values.

4. Optionally implement a blink “mask” sequence to indicate portions of the template to be ignored during comparisons, e.g. near transitions. This may allow for a user to deviate from a desired “blink sequence,” such as a plus or minus one (1) error window, wherein one or more of lens activation, control, and focus change can occur. Additionally, this may allow for variation in the user's timing of the blink sequence.

A blink sequence may be defined as follows:

1. blink (closed) for 0.5 s

2. open for 0.5 s

3. blink (closed) for 0.5 s

At a one hundred (100) ms sample rate, a twenty (20) sample blink template is given by blink_template=[1,1,1, 0,0,0,0,0, 1,1,1,1,1, 0,0,0,0,0, 1,1]. The blink mask is defined to mask out the samples just after a transition (0 to mask out or ignore samples), and is given by blink_mask =[1,1,1, 0,1,1,1,1, 0,1,1,1,1, 0,1,1,1,1, 0,1]. Optionally, a wider transition region may be masked out to allow for more timing uncertainty, and is given by blink_mask=[1,1,0, 0,1,1,1,0, 0,1,1,1,0, 0,1,1,1,0, 0,1].

Alternate patterns may be implemented, e.g. single long blink, in this case a 1.5 s blink with a 24-sample template, given by blink_template=[1,1,1,1,0,0, 0,0,0,0,0,0, 0,0,0,0,0,0, 0,1,1,1,1,1]. A further alternative pattern may be implemented as indicative of sleep, in this case a 2.4 s blink (or eyes that have closed for sleep) with a 24-sample template, given by blink_template =[0,0,0,0,0,0, 0,0,0,0,0,0, 0,0,0,0,0,0, 0,0,0,0,0,0].

In an alternative embodiment, this blink_template is used without a blink_mask. It is important to note that the above example is for illustrative purposes and does not represent a specific set of data.

Detection may be implemented by logically comparing the history of samples against the template and mask. The logical operation is to exclusive-OR (XOR) the template and the sample history sequence, on a bitwise basis, and then verify that all unmasked history bits match the template. For example, as illustrated in the blink mask samples above, in each place of the sequence of a blink mask that the value is logic 1, a blink has to match the blink mask template in that place of the sequence. However, in each place of the sequence of a blink mask that the value is logic 0, it is not necessary that a blink matches the blink mask template in that place of the sequence. For example, the following Boolean algorithm equation, as coded in MATLAB®, may be utilized.

matched=not (blink_mask)|not (xor (blink_template, test_sample)),

wherein test sample is the sample history. The matched value is a sequence with the same length as the blink template, sample history and blink_mask. If the matched sequence is all logic 1's, then a good match has occurred. Breaking it down, not (xor (blink_template, test_sample)) gives a logic 0 for each mismatch and a logic 1 for each match. A logic OR with the inverted mask forces each location in the matched sequence to a logic 1 where the mask is a logic 0. Accordingly, the more places in a blink mask template where the value is specified as logic 0, the greater the margin of error in relation to a person's blinks is allowed. MATLAB® is a high-level language and implementation for numerical computation, visualization and programming and is a product of MathWorks, Natick, Mass. It is also important to note that the greater the number of logic 0's in the blink mask template, the greater the potential for false positive matched to expected or intended blink patterns. It should be appreciated that a variety of expected or intended blink patterns may be programmed into a device with one or more active at a time and in at least one embodiment control the use of particular blink patterns to be used in a particular operation state. More specifically, multiple expected or intended blink patterns may be utilized for the same purpose or functionality, or to implement different or alternate functionality. For example, one blink pattern may be utilized to cause the lens to change operation state between at least an asleep operation state and an awake operation state. The blink detection in at least one embodiment also can detect when the eyelids remain closed, which would be detected as a continuous blink; the eyelids have a movement trajectory to closing for sleep, which would be detected as a partial blink or series of partial blinks such as when a portion of the ultrasound modules (or transducers) are covered by an eyelid after a blink has occurred; and eyelid droop, which would be detected as a change in the steady state position of the upper and/or lower eyelid from its normal steady state position with or without confirmation of gaze position and/or head droop.

In at least one embodiment, the ultrasound module 110 returns a logic 1 to the system controller 130 when a reflected sound pressure wave is received within a predetermined time of the sound pressure wave being transmitted by the transducer, but when no reflected sound pressure wave is detected within that predetermined time, then the ultrasound module 110 returns a logic 0 to the system controller 130. In at least one embodiment, the predetermined time approximates the expected time of flight for the sound pressure wave from the ultrasound module 110 to the eyelid and the resulting reflection back to the ultrasound module 110.

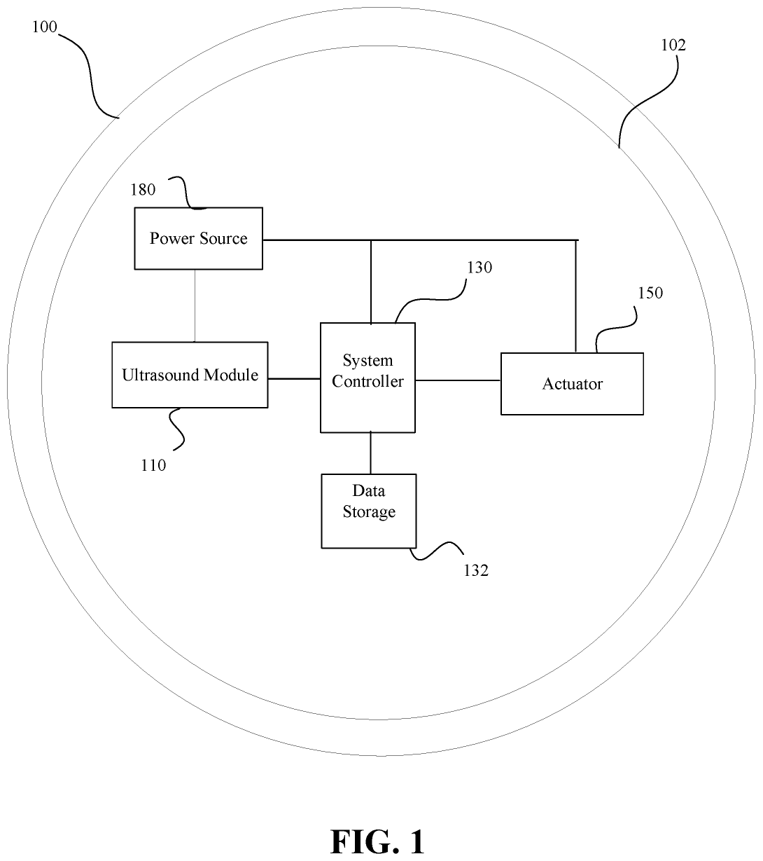

FIGS. 1-8 and 12 illustrate different embodiments according to the invention that include a system controller 130 connected to a timing circuit 140 and an ultrasound module (collectively referred to as 110) that are on a contact lens 100. The ultrasound module 110 may take a variety of forms including distinct transmit and receive transducers or a shared transmit/receive transducer. Depending on a particular implementation, there may be multiple ultrasound modules 110 present on the contact lens to facilitate particular functionality for the contact lens or alternatively multiple transducers connected to one or more ultrasound modules 110. Many of the figures include an actuator 150 as part of the system with the actuator 150 being representative of, for example, lens accommodation components, data collection components, data monitoring components, and/or functional components such as an alarm.

The system controller 130 in at least one embodiment uses at least one predetermined threshold or template for interpreting the output of the ultrasound module(s) 110. In another embodiment, the system controller 130 makes use of at least one template (or pattern) to which a series of outputs of the ultrasound module(s) 110 are compared against to determine whether the template has been satisfied, for example based on a match to the pattern and/or a threshold being met, exceeded or less than resulting in the template being satisfied. In at least one embodiment, the problem template includes only at least one threshold. In an alternative embodiment, both thresholds and patterns are used by the system controller 130 to interpret a received series of sound pressure waves. In at least one embodiment as illustrated in FIG. 1, the system controller 130 is in electrical communication with a data storage 132 that stores the threshold(s) and/or template(s). In at least one embodiment, a plurality of templates includes any combination of patterns and thresholds. Examples of data storage 132 include memory such as persistent or non-volatile memory, volatile memory, and buffer memory, a register(s), a cache(s), programmable read-only memory (PROM), programmable erasable memory, magneto resistive random-access memory (RAM), ferro-electric RAM, flash memory, and polymer thin film ferroelectric memory. In an alternative embodiment, the output(s) of the ultrasound module 110 to the system controller 130 is converted by the system controller 130 into data (or a signal(s)) for control of the actuator 150. In an alternative embodiment, the system controller 130 interprets the output of the ultrasound module 110 using a predefined protocol.

FIG. 1 illustrates a system on a contact lens 100 having an electro-active region 102 with an ultrasound module 110, a system controller 130, an actuator 150, and a power source 180. In at least one further embodiment, the electro-active region 102 includes an electronics ring around the contact lens 100 on which the electronics are located. The ultrasound module 110 in at least one embodiment has two-way communication with the system controller 130. The actuator 150 receives an output from the system controller 130. In at least one alternative embodiment, the actuator 150 is omitted from one or more of the illustrated embodiments in this disclosure.

The actuator 150 may include any suitable device for implementing a specific function based upon a received command signal from the system controller 130. For example, if a set of data samples matches a template, the system controller 130 may enable the actuator 150 to change focus of the contact lens, provide an alert to the wearer such as a light (or light array) to pulse a light or cause a physical wave to pulsate into the wearer's retina (or alternatively across the lens), or to log data regarding the state of the wearer. Further examples of the actuator 150 acting as an alert mechanism include an electrical device; a mechanical device including, for example, piezoelectric devices, transducers, vibrational devices, chemical release devices with examples including the release of chemicals to cause an itching, irritation or burning sensation, and acoustic devices; a transducer providing optic zone modification of an optic zone of the contact lens such as modifying the focus and/or percentage of light transmission through the lens; a magnetic device; an electromagnetic device; a thermal device; an optical coloration mechanism with or without liquid crystal, prisms, fiber optics, and/or light tubes to, for example, provide an optic modification and/or direct light towards the retina; an electrical device such as an electrical stimulator to provide a mild retinal stimulation or to stimulate at least one of a corneal surface and one or more sensory nerves of the cornea; or any combination thereof. In an alternative embodiment, the actuator 150 sends an alert to an external device using, for example the ultrasound module 110. The actuator 150 receives a signal from the system controller 130 in addition to power from the power source 180 and produces some action based on the signal from the system controller 130. For example, if the output signal from the system controller 130 occurs during one operation state, then the actuator 150 may alert the wearer that a medical condition has arisen or the contact lens is ending/nearing its useful life and/defective. In an alternative embodiment, the actuator 150 delivers a pharmaceutical product to the wearer in response to an instruction from the system controller 130. In an alternative embodiment, the signal outputted by the system controller 130 during another operation state, then the actuator 150 will record the information in memory for later retrieval. In a still further alternative embodiment, the signal will cause the actuator 150 to alarm and store information. In an alternative embodiment, the system controller 130 stores the data in the memory (e.g., data storage 132 in other embodiments) associated with the system controller 130 and does not use the actuator 150 for data storage and in at least one embodiment, the actuator 150 is omitted. As set forth above, the powered lens of the present invention may provide various functionality; accordingly, one or more actuators may be variously configured to implement the functionality.

FIG. 1 also illustrates a power source 180, which supplies power for numerous components in the system. The power may be supplied from a battery, energy harvester, or other suitable means as is known to one of ordinary skill in the art. Essentially, any type of power source 180 may be utilized to provide reliable power for all other components of the system. In an alternative embodiment, communication functionality is provided by an energy harvester that acts as the receiver for the time signal, for example in an alternative embodiment, the energy harvester is a photovoltaic cell, a photodiode(s), or a radio frequency (RF) receiver, which receives both power and a time-base signal (or indication). In a further alternative embodiment, the energy harvester is an inductive charger, in which power is transferred in addition to data such as RFID. In one or more of these alternative embodiments, the time signal could be inherent in the harvested energy, for example N*60 Hz in inductive charging or lighting.

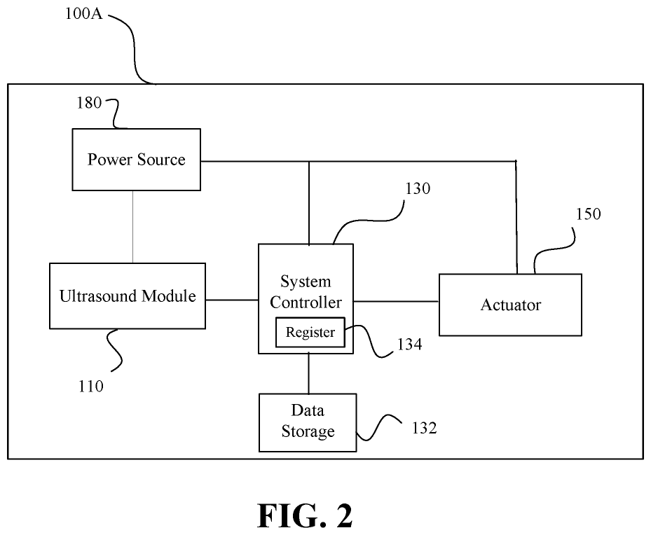

In at least one embodiment as illustrated in FIG. 2, the contact lens 100A includes the system controller 130 having a register 134 for storing data samples from the ultrasound module 110. In a further embodiment, there is an individual register for each ultrasound module 110 and/or a receiving transducer present on the contact lens 100A. The use of a register 134 in at least one embodiment allows for the comparison of data with prior data, a threshold, a preset value, a calibrated value, a target processing value, or a template with or without a mask. In an alternative embodiment, other data storage is used instead of a register(s). In an alternative embodiment, the register 134 is part of the data storage 132.

Based on this disclosure, it should be appreciated that in addition to the presence of the ultrasound module 110 on the contact lens 100 that additional sensors may be included as part of the contact lens to monitor characteristics of the eye and/or the lens. In at least one embodiment, at least a portion of the actuator 150 is consolidated with the system controller 130.

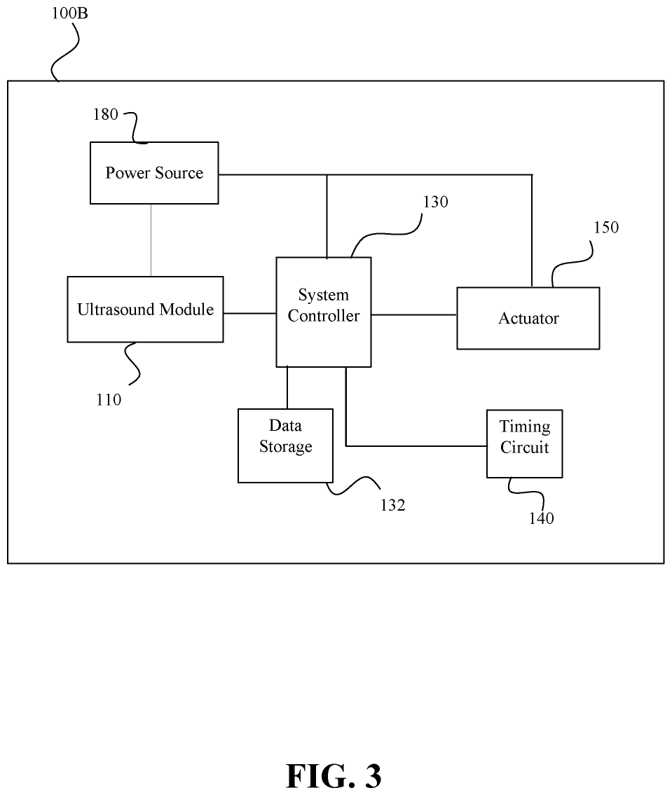

FIG. 3 illustrates another contact lens 100B that adds a timing circuit 140 to the system illustrated in FIG. 1. In an alternative embodiment, the timing circuit 140 may also be added to the embodiment illustrated in FIG. 2. The timing circuit 140 provides a clock function for operation of the contact lens. As illustrated, the timing circuit 140 is connected to the system controller 130. In at least one embodiment, the timing circuit 140 drives the system controller 130 to send a signal to the ultrasound module 110 to perform a function based on a sampling time interval, which in at least one embodiment is variable based on the output from the ultrasound module 110 to the system controller 130. In an alternative embodiment, the timing circuit 140 is part of the system controller 130.

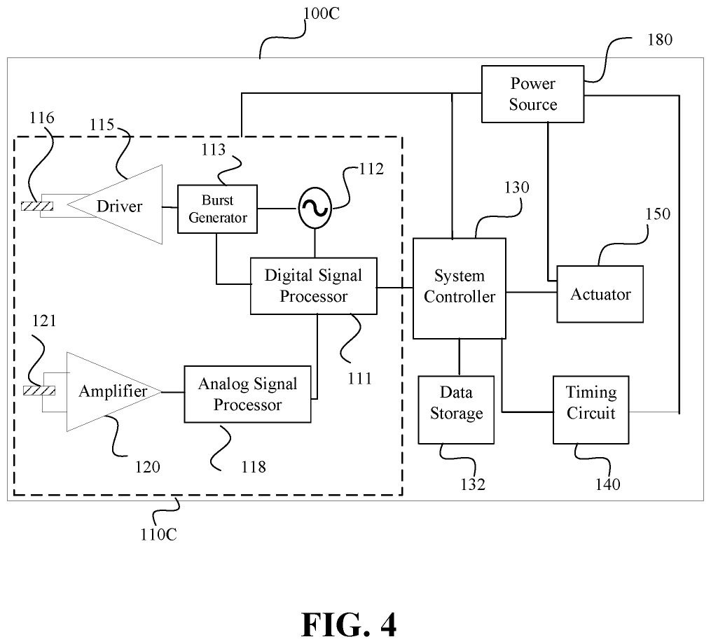

FIGS. 4-8 and 12 illustrate different ultrasound modules that illustrate different transmit paths and receive paths examples of paths that facilitate transmitting and receiving sound pressure waves from one or more transducers 116, 121 that start or end with a processor 111 and/or the system controller 130 depending on the example embodiment.

FIG. 4 illustrates a contact lens 100C that includes an ultrasound module 110C having distinct transmit and receive sides to the ultrasound module 110C. The illustrated ultrasound module 110C includes a digital signal processor 111, an oscillator 112, a burst generator 113, a transmit driver 115, a transmit ultrasound transducer 116, an analog signal processor 118, a receive amplifier 120, and a receive ultrasound transducer 121. In at least one embodiment, the burst generator 13 produces a series of 1's and 0's, which in an alternative embodiment may also be used to facilitate communication with another lens and/or an external device. In at least one embodiment, the burst generator 113 incorporates a unique identifier for the contact lens based on the amplitude, the frequency, the length, and/or the code modulation of the signal. In a further embodiment, the unique identifier is provided by the system controller 130, the digital signal processor 111, the oscillator 112, and/or the burst generator 113. A similar use of unique identifier may be used with other embodiments in this disclosure. In at least one alternative embodiment for the ultrasound module 110C, the digital signal processor 111 is combined with the system controller 130. In another alternative embodiment, the analog signal processor 118 is combined with the digital signal processor 111 and/or replaced with an analog-to-digital convertor as illustrated in a later figure. These two alternative embodiments may be combined to provide a further alternative embodiment.

The digital signal processor 111 receives a control signal from the system controller 130. In at least one embodiment, the digital signal processor 111 includes a resettable counter and a time-to-digital convertor and transmit/receive sequencing controls. The oscillator 112 in at least one embodiment is a switched oscillator. In at least one embodiment, the frequency of the oscillator 112 is programmable through a preset oscillator value, the system controller or external interface (e.g., an interface with an external device). The frequency can be tuned using a reference oscillator and an external interface. In at least one further embodiment, the frequency is set or tuned to a value that minimizes transmit and receive electrical power and allows the transmit ultrasound transducer 116 to produce a sound pressure wave that will have maximum amplitude at the receiver input. In a more particular embodiment, the oscillator 112 is a programmable frequency oscillator such as a current starved ring oscillator where the current and the capacitance control the oscillation frequency where the frequency can be altered by changing the current supplied to the oscillator. In at least one embodiment, the wavelength of the sound pressure wave is tuned based on the dimensions of the transducer used. In a further embodiment, the oscillator 112 varies over time for optimal transmission characteristics. In a still further embodiment, the frequency is calibrated using a reference frequency provided through an external interface and an automatic frequency control (AFC) circuit. The frequency is preset with the AFC tuning it. The frequency can be directly set through the serial interface, which is accessed through the external communications link.

In at least one embodiment, the counter in the digital signal processor 111 begins to count pulses outputted from the oscillator 112. The burst generator 113 gates the oscillator signal for a fixed amount of time defined as the burst length. In at least one embodiment, the burst length is programmable or determined by static timing relationships within the burst generator 113.

The output voltage of the burst generator 113 may be level shifted to the appropriate value for the transmit driver 115 and the transmit ultrasound transducer 116. An example of the transmit ultrasound transducer 116 is a piezoelectric device which converts applied burst voltage to a sound pressure wave. In at least one embodiment, the sound pressure wave includes a burst or multiple sound pressure waves. In a further embodiment, the transmit ultrasound transducer 116 is made of any piezoelectric material that is compatible with the power source and the physical properties of the contact lens. Another example of a transducer is a polyvinylidene fluoride or polyvinylidene difluoride (PVDF) film. The sound pressure wave produced by the transmit ultrasound transducer 116 propagates from the contact lens 100 into the field of view. The speed of sound in air typically is 343 meters/second, so in an embodiment that measures time of flight, then the distance to the object can be measured by dividing the travel time between the propagation of the sound pressure wave and receipt of the reflected sound pressure wave by the receive ultrasound transducer 121.

The receive amplifier 120 and the analog signal processor 118 in at least one embodiment are turned on with the oscillator 112 or turned on after a predetermined delay after the oscillator 112 is started. When there is a predetermined delay, power for contact lens operation may be lowered during the period of delay. In an embodiment where the receive amplifier 120 and the analog signal processor 118 are started with the oscillator 112, the receive amplifier 120 will receive an output from the receive ultrasound transducer 121 proximate to when the sound pressure wave is output by the transmit ultrasound transducer 116. This output from the receive ultrasound transducer 121 can be used to reset the counter in the digital signal processor 111. In a further embodiment, the detection of the transmit sound pressure wave can be used as an indicator that a true transmit signal has been generated.

A sound pressure wave received by the receive ultrasound transducer 121 will produce a voltage signal with an amplitude, a frequency and burst length properties related to the transmitted sound pressure wave. The voltage signal is amplified by the receive amplifier 120 before being sent to the analog signal processor 118, which in an alternative embodiment to embodiments having the receive amplifier 120 and the signal processor 118 are combined into a signal processor. The analog signal processor 118 may include, but is not limited to, frequency selective filtering, envelope detection, integration, level comparison and/or analog-to-digital conversion. Based on this disclosure, it should be appreciated that these functions may be separated into individual blocks with some examples being illustrated in later figures. The analog signal processor 118 produces a received signal that represents the received sound pressure wave at the receive ultrasound transducer 121, which in implementation will have a slight delay. The received signal is passed from the analog signal processor 118 to the digital signal processor 111. When transmission time is used, the digital signal processor 111 will stop the counter that is counting pulses from the oscillator 112 when the received signal is received. In such an embodiment, the measured time can be compared to a predetermined value or if a preset time limit has been reached to determine whether the eyelid is closed or at least covering the transducer. In other embodiments where communication may also be occurring, the digital signal processor 111 interprets the received signal for a message from, for example, the other contact lens or an external device. The resulting output from the digital signal processor 111 is provided to the system controller 130.

In an alternative embodiment, the discussed embodiments in this disclosure make use of the voltage signal amplitude to determine if a blink is detected. If the amplitude is above a predetermine (or preset) strength threshold such as 0.025% of the voltage amplitude used to generate the sound pressure wave. In at least one embodiment, if the voltage signal amplitude for the received sound pressure wave is below the threshold, then the ultrasound module 110 discards the signal with no more processing as it means a blink was not detected. In other embodiments, the ultrasound module 110 may pass the amplitude information to the system controller 130 for the system controller 130 to make the determination. In at least one embodiment, the time of flight is not used to determine a blink. In at least one embodiment, the amplitude detection is done as part of a time of flight analysis including as providing a second input for determining whether a blink occurred.

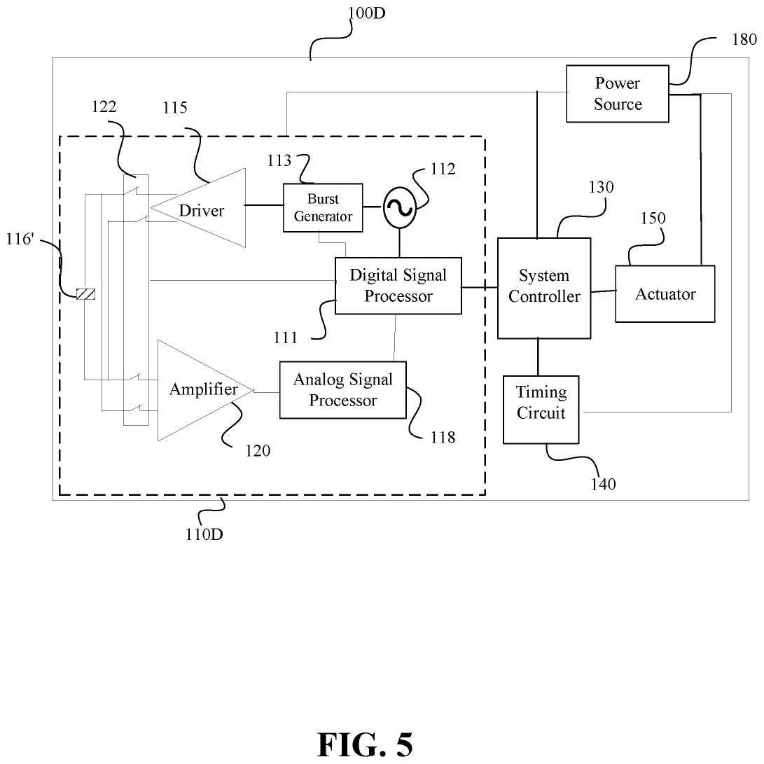

FIG. 5 illustrates a contact lens 100D with an ultrasound module 110D. The illustrated ultrasound module 110D includes one ultrasound transducer 116′ that is shared by the transmit and receive sides (or paths), which may be implemented in the other embodiments. The single ultrasound transducer 116′ is multiplexed between transmit and receive operation through use of a switch 122. The digital signal processor 111D uses the output of the burst generator 113 to switch the transducer 116′ to transmit mode by connecting the transmit driver 115 to the transducer 116′. When the burst is completed, then the digital signal processor 111D switches the switch 122 to the receive mode by connecting the receive amplifier 120 to the transducer 116′. One advantage to this configuration is that the transducer area is reduced from two transducers to one transducer, but a drawback to this configuration is that a short time of flight may not be detected or if the ultrasound module is being used for communication, then a received communication may be missed during a transmission or vice versa. In a least one embodiment, the transducer 116′ is made from PVDF to minimize (and possibly eliminate) the ringing time after generating the sound pressure wave. As with the previous embodiment, a delay may be imposed after transmission before the receive amplifier 120 is powered. The remaining components of the illustrated embodiment remain the same from the embodiment illustrated in FIG. 4.

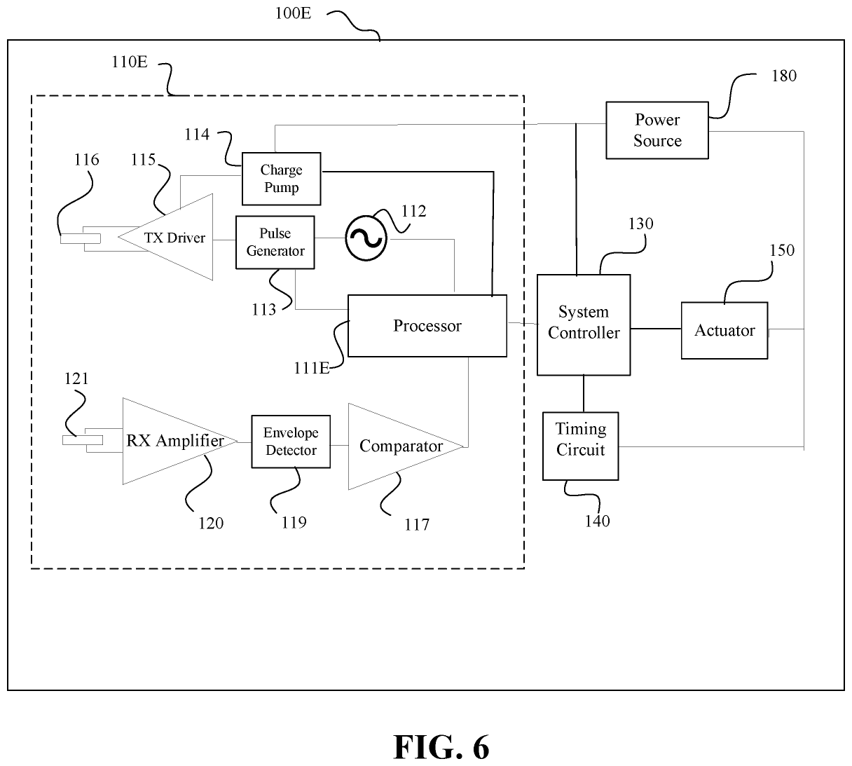

FIG. 6 illustrates a contact lens 100E with an ultrasound module 110E. The illustrated ultrasound module 110E includes a processor 111E, the oscillator 112, the pulse generator 113, a charge pump 114, the transmit driver 115, the transmit ultrasound transducer 116, a comparator 117, an envelope detector 119, the receive amplifier 120, and the receive ultrasound transducer 121. The charge pump 114 is electrically connected to the power source 180 and to the transmit driver 115, which provides a voltage to the transmit ultrasound transducer 116 to create the sound pressure wave to be emitted by the transducer 116. In at least one embodiment, the transmit driver 115 includes an inverter or an H-bridge, and in further embodiments includes an output driver circuit. In at least one embodiment, the charge pump 114 increases the voltage through the relationship between charge and capacitance with voltage by increasing the charge on a capacitance component(s) (e.g., a capacitor). The voltage output from the charge pump 114, in at least one embodiment, is used as the supply voltage to the transmit driver 115. The transmit driver 115 switches between the output of the charge pump 114 and ground in an alternating fashion in response to the input from the pulse generator 113 to produce an alternating voltage. The alternating voltage is applied by the driver 115 to polarize the material of the transducer 116 in one direction and then the other direction to create a mechanical stress causing the material to be displaced in a specific direction (i.e. the direction the transducer is facing). The displacement of the transducer material coupled with the shape and the size of the transducer produce the sound pressure wave. Thus, the larger the applied voltage is to the transducer, the larger the stress and thus the larger the displacement and associated sound pressure wave.

The charge pump 114 is also electrically connected to the processor 111E, which controls operation of the charge pump 114 in at least one embodiment to minimize power consumption by the system, for example by turning off the oscillator 112, the pulse generator 113, and/or the charge pump 114 at times when the ultrasound module 110E does not need to propagate a sound pressure wave. The envelope detector 119 turns the high-frequency output of the receive ultrasound transducer 121 into a new signal that provides an envelope signal representative of the original output signal to be provided to the comparator 117. This illustrated embodiment has the advantage of simplifying the analysis of the output of the receive ultrasound transducer 121 to determine if a particular threshold has been met for the contact lens 100E to perform a function. The comparator 117 provides an output to the processor 111E, which is in electrical communication with the system controller 130.

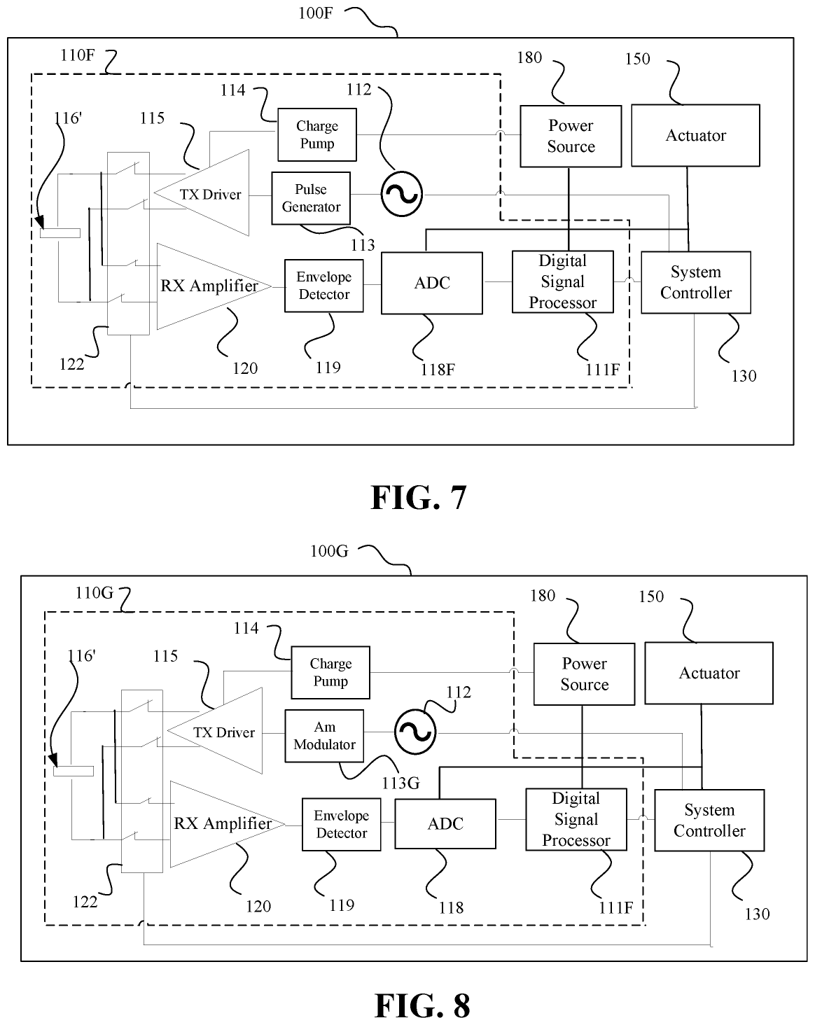

FIG. 7 illustrates a contact lens 100F with an ultrasound module 110F. The illustrated ultrasound module 110F includes a digital signal processor 111F, the oscillator 112, the pulse generator 113, the charge pump 114, the transmit driver 115, the transmit/receive ultrasound transducer 116′, an analog-to-digital converter (ADC) 118F, an envelope detector 119, the receive amplifier 120, and the switch 122. The ADC 118F converts the output from the envelope detector 119 into a digital signal for the digital signal processor 111G.

FIG. 8 illustrates a contact lens 100G with an ultrasound module 110G. The illustrated ultrasound module 110G includes a digital signal processor 111F, the oscillator 112, an amplitude modulation (AM) modulator 113G, the charge pump 114, the transmit driver 115 such as a transmit amplifier, the transmit/receive ultrasound transducer 116′, an analog-to-digital converter (ADC) 118F, an envelope detector 119, the receive ultrasound transducer 121, and the switch 122. In the illustrated embodiment, the charge pump 114, the AM modulator 113G and transmit driver 115 act as the level shifter and the burst generator. The AM modulator 113G in this embodiment is controlled by the digital signal processor 111F. The circuit works where the oscillator signal is provided to the AM modulator 113G, which in at least one embodiment is an AND gate, and the digital signal processor 111F provides a second clock at a frequency much lower than the oscillator frequency. The output of the circuit is then a sequence of pulses that occur during the positive cycle of the lower frequency. The transmit driver 115 has the appropriate gain to output the modulated signal at the charge pump voltage thus providing level shifting.

Based on the disclosure connected to FIGS. 6-8, one of ordinary skill in the art should appreciate that the different ultrasound module configurations and transducer/switch configurations may be interchanged and mixed together in different combinations.

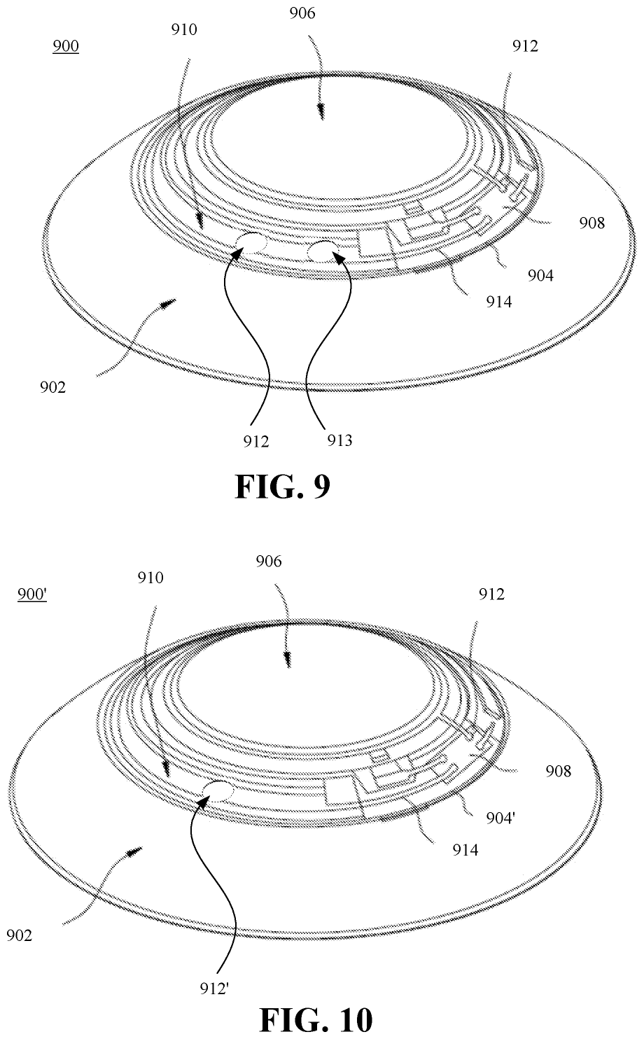

FIG. 9 illustrates a contact lens 900 with an electronic insert 904 having an ultrasound module. The contact lens 900 includes a soft plastic portion 902 which houses the electronic insert 904, which in at least one embodiment is an electronics ring around a lens 906. This electronic insert 904 includes the lens 906 which is activated by the electronics, for example focusing near or far depending on activation (or accommodation level). In at least one embodiment, the electronic insert 904 omits the adjustability of the lens 906. Integrated circuit 908 mounts onto the electronic insert 904 and connects to batteries (or power source) 910, lens 906, and other components as necessary for the system.

In at least one embodiment, a transmit ultrasound transducer 912 and a receive ultrasound transducer 913 are present in the ultrasound module. In at least one embodiment, the integrated circuit 908 includes a transmit ultrasound transducer 912 and a receive ultrasound transducer 913 with the associated signal path circuits. The transducers 912, 913 face outward through the lens insert and away from the eye (i.e., front-facing), and is thus able to send and receive sound pressure waves. In at least one embodiment, the transducers 912, 913 are fabricated separately from the other circuit components in the electronic insert 904 including the integrated circuit 908. In this embodiment, the transducers 912, 913 may also be implemented as separate devices mounted on the electronic insert 904 and connected with wiring traces 914. Alternatively, the transducers 912, 913 may be implemented as part of the integrated circuit 908 (not shown). Based on this disclosure one of ordinary skill in the art should appreciate that transducers 912, 913 may be augmented by the other sensors.

FIG. 10 illustrates another contact lens 900′ with an electronic insert 904′ having an ultrasound module. The contact lens 900′ includes a soft plastic portion 902 which houses the electronic insert 904′. This electronic insert 904′ includes a lens 906 which is activated by the electronics, for example focusing near or far depending on activation (or accommodation level). In at least one embodiment, the electronic insert 904′ omits the adjustability of the lens 906. Integrated circuit 908 mounts onto the electric insert 904′ and connects to batteries (or power source) 910, lens 906, and other components as necessary for the system. The ultrasound module includes a transmit/receive ultrasound transducer 912′ with the associated signal path circuits. The transducer 912′ faces outward through the lens insert and away from the eye, and is thus able to send and receive sound pressure waves. As discussed above, the transducer 912′ may be fabricated separately from the other electronic components prior to mounting on the electronic insert 904 or alternatively implemented on the integrated circuit 908 (not shown). The transducer 912′ may also be implemented as a separate device mounted on the electronic insert 904′ and connected with wiring traces 914. Based on this disclosure one of ordinary skill in the art should appreciate that transducer 912′ may be augmented by the other sensors.

In a further embodiment to the embodiments illustrated in FIGS. 9 and 10, the integrated circuit 908, the power source 910 and the transducers 912, 912′, 913 are present in an area of the contact lens contained in an overmold, which is a material (such as plastic or other protective material) encapsulating the electronic insert 904. In at least one embodiment, the overmold encapsulates the ultrasound module(s).

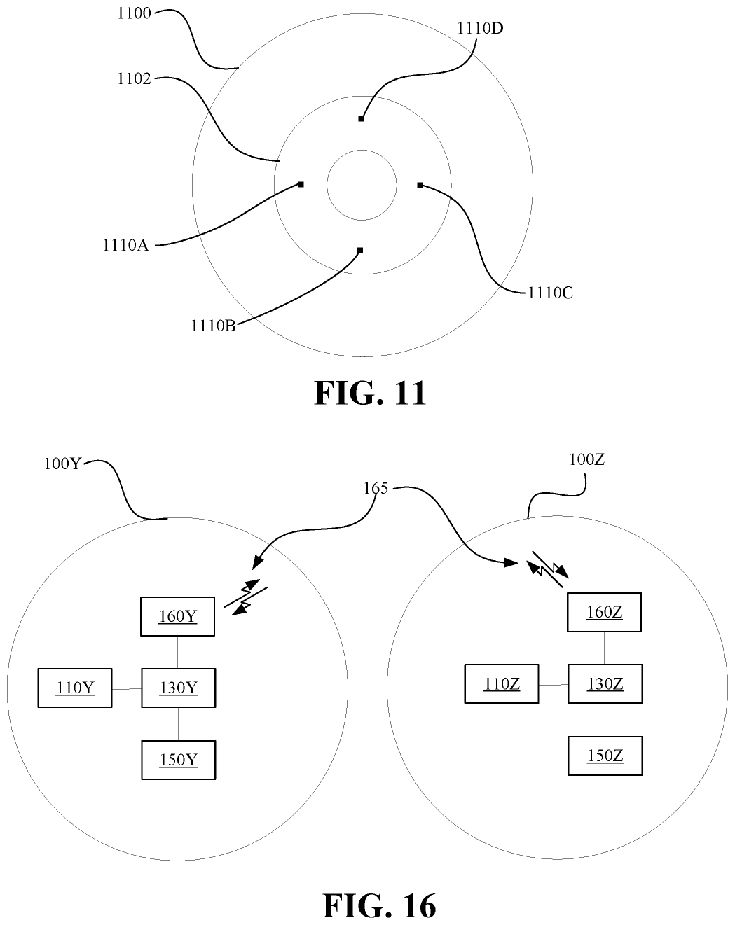

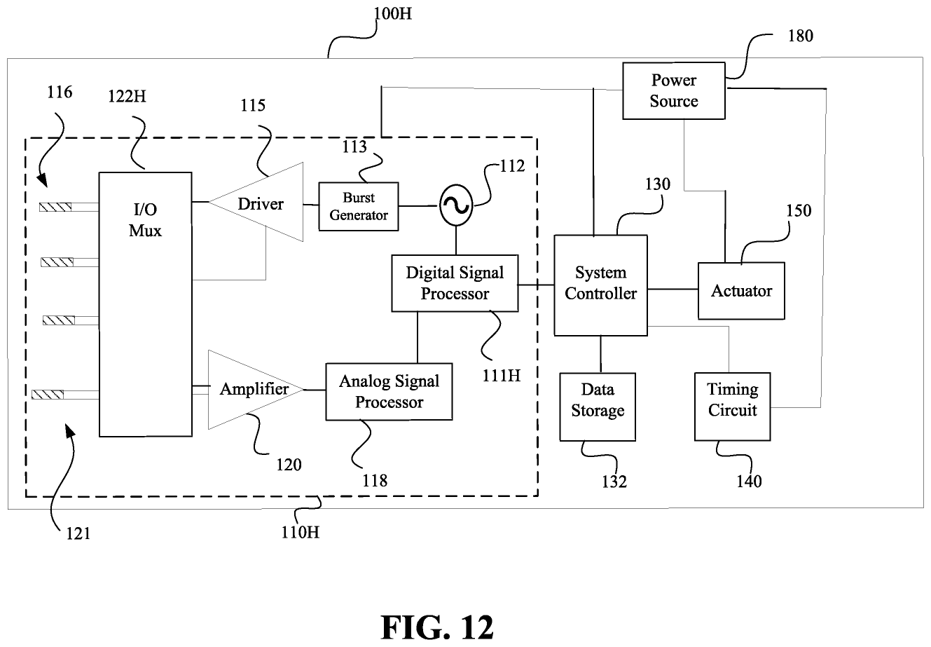

In at least one embodiment as illustrated in FIG. 11 (omits the other components to facilitate presentation clarity), there are a plurality of ultrasound modules 1110A-1110D spaced around the contact lens 1102 on the eye 1100 to increase the fidelity of blink detection. Although four ultrasound modules 1110A-1110D are illustrated, it should be appreciated based on this disclosure that a variety of numbers of ultrasound modules may be used with example numbers of ultrasound modules being any number between 2-8, a plurality of ultrasound modules, and at least one ultrasound module. The illustrated ultrasound modules 1110A-1110D are evenly spaced around the periphery of the contact lens 1102 where evenly spaced includes equal distance between the ultrasound modules (i.e., the same distance between neighboring ultrasound modules) and/or balanced about a diameter drawn through the contact lens 1102. In a further embodiment, the illustrated ultrasound modules are replaced by transducers that are multiplexed together as illustrated in FIG. 12.

In an alternative embodiment illustrated in FIG. 12, the contact lens 100H has one ultrasound module 110H having a plurality of transducers 116, 121 and an I/O multiplexer (mux) 122H attaching the transducers 116, 121 to the ultrasound module components discussed in the above embodiments. FIG. 12 illustrates the inclusion of the digital signal processor 111, the oscillator 112, the burst generator 113, the driver 115, the amplifier 120, and the analog signal processor 118. In an alternative embodiment, these ultrasound module components may be replaced by components from the other described ultrasound module embodiments including using just the transmit or receive paths of those embodiments. An advantage of this configuration is that it reduces the power requirements and weight considerations by eliminating duplicative components and allowing the ultrasound module to drive multiple transmit transducers and to receive analog signals from multiple receive transducers. In at least one embodiment, the transmit transducers and the receive transducers are distributed about the contact lens as discussed above in connection with FIG. 11. In a further embodiment, the transmit transducers and the receive transducers are grouped together in one area of the contact lens.

In at least one embodiment where the contact lens includes rotational stability features, then the number of ultrasound modules is one.

In a further embodiment for contact lenses that have a plurality of ultrasound modules or at least transmit/receive/transceiver transducers, the method includes having the system controller determine which ultrasound module/transducer provides the best blink detection. The system controller selects the ultrasound module/transducer that produces a varying signal as an output response to the sound pressure wave produced by the contact lens, because a varying signal represents the detection of a blink while a continuous output signal represents detection of a sound pressure wave and indicates the ultrasound module may be covered by an eyelid due to the ultrasound module's location relative to the eye. In at least one embodiment, the selected ultrasound module/transducer is the one that has the most varied signal over time when compared to the other ultrasound modules/transducers. This measurement may be made during performance of the above-described blink detection methods. The system controller will deactivate the ultrasound module(s)/transducer(s) that were not selected (i.e., provided a continuous indication of a blink). One benefit to this method is that as the contact lens rotates on the eye, the system controller can change the used ultrasound module/transducer for intra-contact communication.

In a further embodiment for contact lenses that have a plurality of ultrasound modules or at least transmit/receive/transceiver transducers, the method includes having the system controller determine which ultrasound module/transducer provides the best response. The system controller selects the ultrasound module/transducer that produces a highest output response to reflected sound pressure waves.

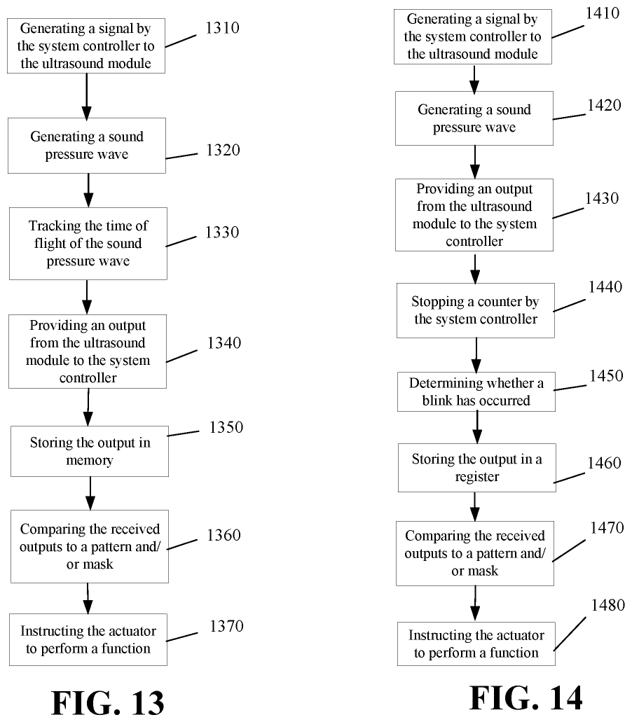

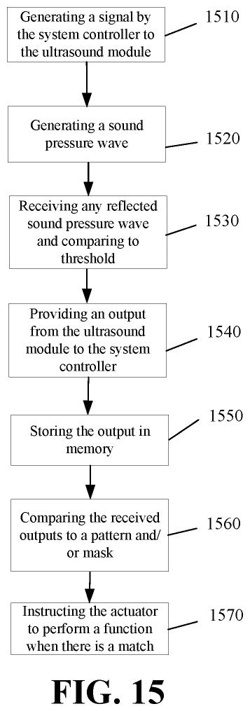

FIGS. 13-15 illustrate methods for determining blinks by using ultrasound time of flight measurements or signal amplitude. FIG. 13 illustrates a method where the blink determination is made by the ultrasound module 110, while FIG. 14 illustrates a method where the blink determination is made by the system controller 130. FIG. 15 illustrates a method where the blink determination is made based on the output signal amplitude. In at least one alternative embodiment, the time of flight measurements and the output signal amplitude are used together to determine the occurrence of a blink. FIG. 13 illustrates a method that begins with the system controller 130 generating a signal to at least one ultrasound module 110 to produce a sound pressure wave, 1310. The at least one ultrasound module 110 generates a sound pressure wave with a transducer facing outward from the eye, 1320. The at least one ultrasound module 110 tracks the passage of time until receipt of a reflected sound pressure wave or the expiration of a predetermined sampling period, 1330. One way to track the time is by use of a counter on either the ultrasound module 110, for example the processor, or, alternatively, the system controller 130. Examples of the predetermined sampling period include 29 μs, 30 μs, 35 μs, and 50 μs. Further examples of the predetermined sampling period include any time within a range of 29 μs to 50 μs (in alternative embodiments including one or both of the endpoints), a range of 30 μs to 50 μs (in alternative embodiments including one or both of the endpoints), and a range of 35 μs to 50 μs (in alternative embodiments including one or both of the endpoints).

In at least one embodiment and as discussed above, when the voltage signal amplitude of the reflected wave is received before expiration of the predetermined sampling period, then it will be treated as a blink having occurred. One benefit is that the counter can run a slower rate and conserve energy for the system by having the trip be receipt of the reflected wave within a predetermined sampling period. In at least one embodiment, the counter runs at a slower rate than that provided by the oscillator and/or the timing circuit.

In an alternative embodiment, the system has a counter running at a high frequency until the predetermined sampling period is reached to see if a reflected wave is detected by the ultrasound module 110. When the predetermined sampling period expires and no blink has been detected, then the ultrasound module 110 determines no blink occurred. An example frequency for the timing circuit and/or the oscillator on which the counter runs is 6-10 MHz to allow for oversampling.

In a further alternative embodiment, there is a second counter running at a higher frequency than a first counter. The second counter runs until at least one of a reflected wave is detected or a predetermined sampling period is reached, based on the outcome the ultrasound module 110 determines if a blink occurred. The first counter, which is running at a slower rate, is used to track time for a reflected wave to come back from something beyond the eyelid for a time of flight measurement and will continue to run even if a blink is detected until a time of flight sampling period has been reached, then the first counter will terminate. The time of flight sampling period, in at least one embodiment, is equivalent to the time it takes for an ultrasound signal to travel to an object at the accommodation point for the wearer (including any margin for hysteresis) and return to the contact lens. In a further embodiment, the first counter will also terminate if a reflected signal is detected between the predetermined sampling period and the time of flight sampling period.

An alternative to the last four embodiments, is that once a blink is detected based on receipt of a reflected wave within the predetermined sampling period, the counter stops tracking the passage of time.

In at least one embodiment, the output from the ultrasound module 110 is binary or a pulse data signal. In a further embodiment, a 0 is used when the time expires before receiving a reflected sound pressure wave and a 1 is used when a reflected sound pressure wave is received prior to the time expiration. In a further embodiment, the sound pressure wave includes a plurality of waves that provide an identifier such that a reflected sound pressure wave can be determined to match the most recently produced sound pressure wave.

The ultrasound module 110 provides an output to the system controller 130, 1340. The system controller 130 in at least one embodiment optionally stores the output in memory including in a further embodiment storing the output in a register, 1350. The system controller 130 comparing the recently received outputs to a pattern and/or mask to determine if the wearer has provided instructions or a condition is detected based upon the pattern and/or mask that is matched, 1360. In at least one further embodiment, the system controller 130 sending an instruction signal to the actuator 150 to perform a function, 1370.

FIG. 14 illustrates a method that begins with the system controller 130 generating a signal to at least one ultrasound module 110 to produce a sound pressure wave, 1410. The at least one ultrasound module 110 generates a sound pressure wave with a transducer facing outward from the eye and sends a signal to the system controller 130 that a sound pressure wave has been emitted, 1420. The at least one ultrasound module 110 produces an output to the system controller 130 when a reflected sound pressure wave is received at a transducer, 1430. The system controller 130 stops a counter upon receipt of the signal from the at least one ultrasound module 110, 1440. In at least one embodiment, the system controller 130 delays the start of the counter to account for approximately the time for the ultrasound module 110 to perform its processing. In an alternative embodiment, the ultrasound module 110 sends a signal to the system controller 130 to start the counter. When the counter has a time less than a predetermined time (or predetermined sampling period or time), the system controller 130 determines a blink has occurred, 1450. In an alternative embodiment, the at least one ultrasound module 110 provides a signal including the time of flight instead of the system controller 130 tracking the time of flight. In another alternative embodiment, the system controller 130 providing a termination signal to the at least one ultrasound module 110 when the counter reaches or exceeds the predetermined sampling period (or time). The termination signal providing instruction to the at least one ultrasound module 110 to turn-off the receive path for detecting the reflected sound pressure wave. In at least one embodiment, the termination signal provides the benefit of conserving power. In a further embodiment, the sound pressure wave includes a plurality of waves that provide an identifier such that a reflected sound pressure wave can be determined to match the most recently produced sound pressure wave.

The system controller 130 in at least one embodiment stores the output in memory including in a further embodiment storing the output in a register, 1460. The system controller 130 comparing the recently received outputs to a pattern and/or mask to determine if the wearer has provided instructions or a condition is detected based upon the pattern and/or mask that is matched, 1470. In at least one further embodiment, the system controller 130 sending an instruction signal to the actuator 150 to perform a function, 1480.

FIG. 15 illustrates a method that begins with the system controller 130 generating a signal to at least one ultrasound module 110 to produce a sound pressure wave, 1510. The at least one ultrasound module 110 generates a sound pressure wave with a transducer facing outward from the eye and sends a signal to the system controller 130 that a sound pressure wave has been emitted, 1520. The at least one ultrasound module 110 receives any reflected sound pressure wave and compares it to a threshold, 1530. In an alternative embodiment, the at least one ultrasound module terminates the waiting for receipt of a reflected sound pressure wave upon an expiration of a predetermined sampling period. In such an embodiment, then the time periods and ranges discussed in connection with FIG. 13 may be used. In at least one embodiment, the output from the ultrasound module 110 is binary or a pulse data signal. In a further embodiment, a 0 is used when no sound pressure wave is detected and/or the time expires before receiving a reflected sound pressure wave and a 1 is used when a reflected sound pressure wave is received at any time or prior to the time expiration. In a further embodiment, the sound pressure wave includes a plurality of waves that provide an identifier such that a reflected sound pressure wave can be determined to match the most recently produced sound pressure wave.

The ultrasound module 110 provides an output to the system controller 130, 1540. The system controller 130 in at least one embodiment optionally stores the output in memory including in a further embodiment storing the output in a register, 1550. The system controller 130 comparing the recently received outputs to a pattern and/or mask to determine if the wearer has provided instructions or a condition is detected based upon the pattern and/or mask that is matched, 1560. In at least one further embodiment, the system controller 130 sending an instruction signal to the actuator 150 to perform a function when there is a match, 1570.

In an alternative embodiment to the embodiments illustrated in FIGS. 13-15, steps 1350-1370, 1460-1480, and 1550-1570 are omitted and/or supplemented by the system controller 130 disregarding the output of the ultrasound module 110 when a blink is detected. In a further embodiment, the system controller 130 changes operation of the contact lens when a blink is detected.

In a further embodiment to any of the above method embodiments, when eyelid closure is detected over a predetermined number of consecutive sampling periods based on blinks being determined, the system controller 130 will lengthen the time between samplings. For example, after one minute of samples, decreasing the sampling frequency (or rate) by half. When the blink determination returns no blink (i.e. the eyelids are open), then the system controller 130 returns the sampling frequency to the normal rate. In an alternative embodiment, the system controller 130 may have a plurality of sampling frequencies that allow for a further slowing down of the samplings the longer the continuous detection of blinks occurs, for example when the person may be asleep and continues to be asleep. One benefit of this is that it will reduce the power consumption relating to making a blink determination. In an alternative embodiment, the system controller 130 may change operation states between a wake operation state and an asleep operation state where each operation state has its own set of parameters such as sampling frequency or sound pressure wave characteristics. The system controller 130 would change between operation states based on recorded information that would, for example, indicate the wearer was awake or asleep. In an embodiment, where there are multiple ultrasound modules 110 and/or a plurality of multiplexed transducers, the sleep determination is made when all data points agree that a blink has occurred. In a further embodiment, when there is communication between the contact lenses, both contact lenses will need to have reached the same determination of whether the wearer is awake or asleep.

Alternatively, when a series of consecutive blinks is detected, the system controller 130 calibrates the contact lens by determining the amount of time needed for the sound pressure wave to travel from the ultrasound module 110 to the eyelid and be reflected back to the ultrasound module 110. The time measurement then can be used for the predetermined blink threshold for a time of flight measurement. In a further embodiment, a predetermined time buffer is added to the predetermined blink threshold. In at least one embodiment, the time buffer is a fixed amount of time or a percentage of the detected time measurement.

In a further embodiment, a pair of contact lenses 100Y, 100Z illustrated in FIG. 16 work in conjunction together to confirm that a blink has been detected by both contact lenses 100Y, 100Z. Time measurement signals (or time-of-flight measurement signals) are generated by at least one ultrasound module 110Y, 110Z on each contact lens 100Y, 100Z, respectively. In an alternative embodiment, the time measurement signal represents a distance. The time measurement signals are transmitted to the system controller 130 on one of the contact lenses 100Y/100Z, for example the system controller 130Z on the second contact lens 100Z (one of ordinary skill in the art should appreciate based on this disclosure that the first and second contact lenses could be switched or an external device could be used to perform the processing discussed in this method). In the example, the ultrasound module 110Z on the second contact lens 100Z sends the time measurement signal to the system controller 130Z and the ultrasound module 110Y on the first contact lens 100Y sends its time measurement signal to the system controller 130Y that sends the time measurement signal over a communications link 165 established between the communications modules 160Y, 160Z (or alternatively using ultrasound modules 110X, 110Y) to the system controller 130Z on the second contact lens 100Z. In an alternative embodiment, instead of or in addition to the time measurement signal, an amplitude value and/or representation is communicated. In an alternative embodiment, the ultrasound modules 110Y, 110Z provide a binary indication of whether a reflected sound pressure wave was detected representing the detection of a blink. As discussed previously, the communications link 165 may be established using the ultrasound modules 130 or the communications module 160. The system controller 130Z stores the blink determinations from both contact lenses or alternatively stores it as a blink only when both contact lenses detected a blink. The system controller 130Z comparing the recently received blink determinations to a pattern and/or mask depending on the results of that comparisons sending or not sending instructions to at least one of the actuators 150Y, 150Z to perform an action.

The determination of a blink by both lenses may be used as part of the lenses' operation to change the accommodation level (or focus) or change how the contact lenses function based on a change to electrical or physical properties from the wearer blinking and/or having their eyelids partially closed. This determination could be used for other purposes by the contact lenses, for example including data collection or determining whether the wearer has fallen asleep or a medical condition has arisen. In a still further embodiment, the detection of a blink by both contact lenses could be used to ignore that particular sound pressure wave sample if the contact lenses are using ultrasound to communicate with each other or an external device, detect the wearer's nose, or determine the time of flight to an object being viewed by the wearer. In addition, the mutual blink determinations can be used as part of other accommodation techniques.

Although shown and described in what is believed to be the most practical embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims.

What is claimed is:

1. An ophthalmic lens system comprising:
an ophthalmic lens;
at least one ultrasound module in the ophthalmic lens, the at least one ultrasound module having at least one transmit transducer and at least one receive transducer;
at least one system controller in electrical communication with the at least one ultrasound module, the at least one system controller configured to provide an initiation signal to the at least one ultrasound module to cause the at least one ultrasound module to produce at least one sound pressure wave, and the at least one system controller configured to receive any signal from the at least one ultrasound module representative of a reflection sound pressure wave; and
wherein the at least one of the system controller and the at least one ultrasound module configured to determine if eyelid closure indicative of blink has occurred based on receipt of the reflection sound pressure wave within a predetermined time period and/or having an amplitude above a predetermined strength threshold, and the at least one system controller configured to act upon a determination that at least one blink has occurred.

2. The ophthalmic lens system according to claim 1, further comprising an actuator in electrical communication with the system controller, the actuator configured to receive an output signal from the system controller, and the actuator configured to at least one of store data at least temporarily and produce an action based on the output signal.

3. The ophthalmic lens system according to claim 1, wherein the at least one ultrasound module is configured to deactivate the at least one receive transducer after a predetermined sampling period.

4. The ophthalmic lens system according to claim 1, wherein a plurality of ultrasound modules is distributed around the periphery of the ophthalmic lens, the distribution of ultrasound modules providing for at least one ultrasound module to be covered by an eyelid closure.

5. The ophthalmic lens system according to claim 1, further comprising a power supply in electrical communication with the at least one system controller.

6. The ophthalmic lens system according to claim 1, wherein the at least one system controller includes a memory storing at least one blink template for use by the at least one system controller based on an operation state of the system controller to determine if a wearer has provided an instruction.

7. The ophthalmic lens system according to claim 6, wherein the at least one system controller is configured to select at least one blink template stored in the memory.

8. The ophthalmic lens system according to claim 7, wherein the memory stores at least one blink mask to allow for the wearer to deviate from a desired intentional blink sequence for blink detection.

9. The ophthalmic lens system according to claim 7, wherein the memory is configured to store at least one blink determination.

10. The ophthalmic lens system according to claim 1, wherein the at least one system controller determines lid closure to occur when the at least one system controller receives an indication of the reflected sound pressure wave from the at least one ultrasound module within at most ≤50 μs from transmission of the sound pressure wave by the at least one transmit transducer.

11. The ophthalmic system according to claim 1, further comprising a second ophthalmic lens; and
wherein the at least one system controller configured to provide at least one output signal to be sent to the second ophthalmic lens in response to a determination that a blink has occurred.

12. The ophthalmic system according to claim 11, wherein the at least one ultrasound module further configured to provide communications to the second ophthalmic lens.

13. An ultrasonic method for blink detection by an ophthalmic lens wherein the ophthalmic lens includes an ultrasound module in electrical communication with a system controller, the method comprising:
creating a pulse to drive a transducer in the ultrasound module;
emitting a sound pressure wave from the transducer;
producing a signal by the ultrasound module in response to any received sound pressure wave within about 50 μS of the sound pressure wave being emitted by the transducer;
determining by at least one of the ultrasound module and the system controller whether the signal produced is within a predetermined blink threshold; and
storing the blink determination in a memory.

14. The method according to claim 13, further comprising comparing stored blinks to at least one of a blink template and a blink mask by the system controller.

15. The method according to claim 13, wherein the memory includes a register for storing a plurality of blink determinations.

16. The method according to claim 13, wherein the ophthalmic lens contains at least two ultrasound modules at different positions around the lens periphery; and further comprising:
activating the ultrasound module with a most varied output signal in response to the sound pressure wave, and
deactivating by the at least one system controller the non-selected ultrasound modules.

17. An ultrasonic method for blink detection by an ophthalmic lens, the method comprising:
creating a sound pressure wave by at least one transducer;
receiving a sound pressure wave by at least one transducer;
producing an output in response to the received sound pressure wave;
comparing a series of outputs to a predefined blink template and/or a mask by at least one of an ultrasound module and a system controller; and
if at least one set of sampled data matches the blink template and/or the mask, instructing an actuator to perform an action by the system controller.

18. The method of claim 17, further comprising changing an operation state of the ophthalmic lens by the system controller when at least one set of outputs matches the predefined blink template and/or mask.

19. The method of claim 17, further comprising comparing the number of blinks, the duration of the blinks in a sampling period, and a time between blinks in the sampling period to a stored set of samples representative of one or more predetermined intentional blink sequences to receive instructions from the wearer.

20. The method of claim 17, wherein there are two ophthalmic lenses, the method further comprising:
communicating at least the detection of a blink from one ophthalmic lens to the other ophthalmic lens; and
determining whether both ophthalmic lenses detected a blink at substantially the same time.
